(19) United States
(12) Patent Application Publication
(10) Pub. No.: US 2022/0118239 A1
(43) Pub. Date: Apr. 21, 2022

(54) TATTOO MARKER

(71) Applicant: ZHENPING HUADING STAINLESS STEEL PRODUCTS CO., LTD, Nanyang City (CN)

(72) Inventor: Hengzhong Wang, Shanghai City (CN)

(21) Appl. No.: 17/099,034

(22) Filed: Nov. 16, 2020

(30) Foreign Application Priority Data
Oct. 20, 2020 (CN) 202011127154.9

(51) Int. Cl. A61M 37/00

(52) U.S. Cl. CPC A61M 37/0076

(57) ABSTRACT

A tattoo marker, including a housing, a drive motor, a transmission mechanism and a tattoo needle, wherein the housing includes a handle part and a drive part which is detachably arranged on a top end of the handle part, the drive motor is arranged inside the drive part, the transmission mechanism is arranged inside the handle part, the tattoo needle has a top end detachably connected to a head end of the handle part, the transmission mechanism is connected between the drive motor and the tattoo needle, and the drive motor drives the tattoo needle to extend longitudinally through the transmission mechanism. The transmission mechanism is arranged inside the handle part, which makes the whole transmission mechanism moved downwards and consequently makes the drive motor in connection with the transmission mechanism moved downwards too, thereby shortening the distance between the drive motor and the tattoo needle.

### CROSS REFERENCE TO RELATED APPLICATION

This application claims the benefit of and takes priority from Chinese Patent Application Serial No. 202011127154.9 filed on Oct. 20, 2020, the contents of which are herein incorporated by reference.

### BACKGROUND OF THE INVENTION

### Field of the Invention

The disclosure relates to the technical fields of tattoo devices, and in particular to a tattoo marker.

### Description of the Related Art

Tattoo is a picture or design that is marked permanently on a person's skin by making small holes in the skin with a needle and filling them with colored ink. Tattoos are done through a tattoo marker. Existing tattoo markers generally include a housing, in which a drive device is arranged; the housing has a head end detachably connected to a disposable tattoo needle the housing includes a drive part and a handle part; the drive device includes a drive motor and a transmission device; the transmission device is arranged between the drive motor and the disposable tattoo needle; the drive motor drives the disposable tattoo needle to extend through the transmission device; the transmission devices of existing tattoo markers generally are arranged in the drive part; the transmission device has a bottom extending downwards to the handle part for contact with a top of the tattoo needle. Such arrangement results in a long space occupied by the transmission device, consequently leading to a long tattoo marker, which is not convenient for a user to hold and meanwhile increases the noise generated during operation.

Therefore, how to solve the problems in existing technologies that the tattoo maker is too long and is not convenient for a user to hold and noises are increased all because the transmission device inside the tattoo marker is too long becomes an urgent technical problem to be solved by the skilled in the art.

### SUMMARY OF THE INVENTION

The disclosure aims to provide a tattoo marker, so as to solve the problems in the prior art that the tattoo maker is too long and is not convenient for a user to hold and noises are increased all because the transmission device inside the tattoo marker is too long.

A number of technical effects that can be produced by some preferred technical schemes among the technical schemes provided by the disclosure are described below in detail.

In order to achieve the above aim, the disclosure provides the following technical scheme.

The disclosure provides a tattoo marker, including a housing, a drive motor, a transmission mechanism and a tattoo needle, wherein the housing includes a handle part and a drive part which is detachably arranged on a top end of the handle part, the drive motor is arranged inside the drive part, the transmission mechanism is arranged inside the handle part, the tattoo needle is detachably arranged on a head end of the handle part, the transmission mechanism is connected between the drive motor and the tattoo needle, and the drive motor drives the tattoo needle to extend longitudinally through the transmission mechanism.

Preferably, the transmission mechanism includes a faceplate, a sliding support and a sliding member, the drive motor has an output shaft arranged downwards vertically, an angle is set between an axial line of the faceplate and an axial line of the output shaft, and a bearing is arranged at a center of the faceplate, the bearing is in connection with the output shaft through a shaft sleeve, both axial lines of the shaft sleeve and the bearing are collinear with the axial line of the faceplate, an axial line of a shaft hole defined on the shaft sleeve is colinear with the axial line of the output shaft, the sliding support is detachably arranged inside the handle part, the sliding member is arranged on the bottom of the sliding support and is sliding connection with the sliding support in vertical direction, the sliding member has a top in contact connection with the faceplate in a locking way, and the sliding member has a bottom in contact connection with the tattoo needle.

Preferably, the sliding member includes a connection plate, a contact rod arranged on an edge of the connection plate and a plurality of first sliding holes, the contact rod and the plurality of first sliding holes are distributed along a circumferential direction of the connection plate, the contact rod is arranged on a top of the sliding member and extends upwards, the first sliding hole runs through the connection plate, the sliding support includes a support plate, the support plate defines a second sliding hole for the contact rod to pass through at a position corresponding to the contact rod, the support plate is provided with a sliding rod at a position corresponding to the first sliding hole, and the sliding rod is in sliding connection with the sliding hole.

Preferably, a bottom center of the sliding member is further provided with an ejector rod extending downwards for contact with the tattoo needle.

Preferably, at least one locking groove is defined on a bottom edge of the faceplate, a plurality of the locking grooves are distributed along a circumferential direction of the faceplate and extending along a radial direction of the faceplate, and a top end of the contact rod is in contact connection with the locking groove.

Preferably, two first sliding holes and two sliding rods are provided, the contact rod and the first sliding holes form a regular triangle around a center point of one same horizontal plane, or the second sliding hole and the sliding rods form a regular triangle around a center point of one same horizontal plane.

Preferably, an elastic piece is arranged where a top of the tattoo needle contacts the ejector rod.

Preferably, the elastic piece is a rubber mat.

Preferably, the top end of the contact rod is in a ball shape, and a cross section of the locking groove is in a circular arc shape.

Preferably, the drive motor is an electric machine or a pneumatic motor.

The tattoo marker provided by the disclosure has the following benefits.

The disclosure provides a tattoo marker, including a housing, a drive motor, a transmission mechanism and a tattoo needle, wherein the housing includes a handle part and a drive part which is detachably arranged on a top end of the handle part, the drive motor is arranged inside the drive part, the transmission mechanism is arranged inside the handle part, the tattoo needle has a top end detachably connected to a head end of the handle part, the transmission mechanism is connected between the drive motor and the tattoo needle, and the drive motor drives the tattoo needle to extend longitudinally through the transmission mechanism. In existing tattoo markers, the transmission mechanisms generally are arranged inside the drive part; the transmission mechanism has a bottom end extending downwards to the handle part for contact with the tattoo needle so as to drive the tattoo needle to extend, such arrangement results in a long space occupied by the transmission mechanism, consequently leading to a long tattoo marker, which is not convenient to operate, furthermore, the transmission mechanism will increase the noise generated during operation. However, according to the tattoo marker provided in the disclosure, the transmission mechanism is arranged inside the handle part, which makes the whole transmission mechanism moved downwards and consequently makes the drive motor in connection with the transmission mechanism moved downwards too, thereby shortening the distance between the drive motor and the tattoo needle, shortening the length of the transmission mechanism, and shortening the overall length of the tattoo marker so that it is more convenient to hold the tattoo marker; furthermore, the noise generated during operation will be reduced after the transmission mechanism is shortened.

It is understandable that the above general descriptions and the below detailed descriptions are exemplary and illustrative merely, without limiting the disclosure.

### BRIEF DESCRIPTION OF THE DRAWINGS

For a better understanding of the embodiments of the disclosure or the technical scheme in the prior art, accompanying drawings needed in the description of the embodiments or the prior art are simply illustrated below. Obviously, the accompanying drawings described below are some embodiments of the disclosure. For the ordinary skill in the field, other accompanying drawings may be obtained according to the structure shown in these accompanying drawings without creative work.

FIG. 1 is an exploded view of a tattoo marker according to an embodiment of the disclosure.

FIG. 2 is a view of FIG. 1 along line A-A according to an embodiment of the disclosure.

FIG. 3 is a connection diagram of a sliding support and a sliding member according to an embodiment of the disclosure.

FIG. 4 is a structure diagram of a sliding member according to an embodiment of the disclosure.

FIG. 5 is a structure diagram of a sliding support according to an embodiment of the disclosure.

FIG. 6 is a connection diagram of a drive motor and a faceplate according to an embodiment of the disclosure.

FIG. 7 is a structure diagram of a top of a faceplate according to an embodiment of the disclosure.

FIG. 8 is a structure diagram of a bottom of a faceplate according to an embodiment of the disclosure.

FIG. 9 is a cross-sectional view of a connection of a faceplate and a bearing and a shaft sleeve according to an embodiment of the disclosure.

Description of reference numbers: 1 represents a drive motor, 2 represents a handle part, 3 represents a drive part, 4 represents a faceplate, 5 represents a bearing, 6 represents a shaft sleeve, 7 represents a connection plate, 8 represents a contact rod, 9 represents a first sliding hole, 10 represents a support plate, 11 represents a second sliding hole, 12 represents a sliding rod, 13 represents an ejector rod, and 14 represents a locking groove.

### DETAILED DESCRIPTION OF THE SEVERAL EMBODIMENTS

The above purpose, technical scheme and advantages of the disclosure will become more clearly understood from the detailed description of the technical scheme of the disclosure. Obviously, the embodiments described hereinafter are simply part embodiments of the disclosure, but all the embodiments. All other embodiments obtained by those skilled in the art based on the embodiments in the disclosure without creative work are intended to be included in the scope of protection of the disclosure.

Hereinafter embodiments are described with reference to accompanying drawings. The embodiments described below have no limitations to the contents of the invention recorded in the claims appended herein. In addition, the entirety of the constitution represented by the following embodiments is not limited to what required by a solution to the invention recorded in the claims appended herein.

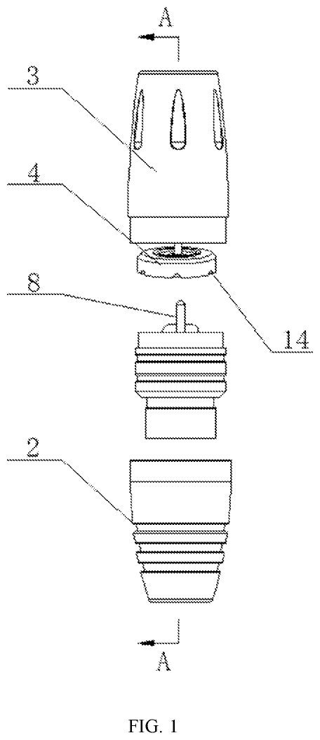

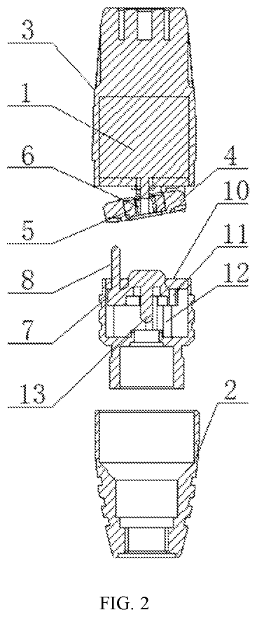

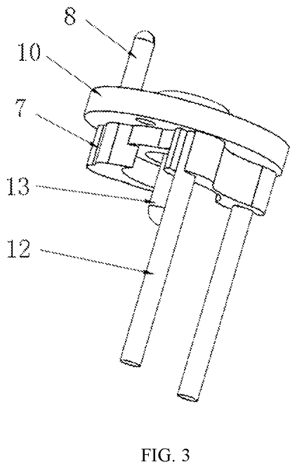

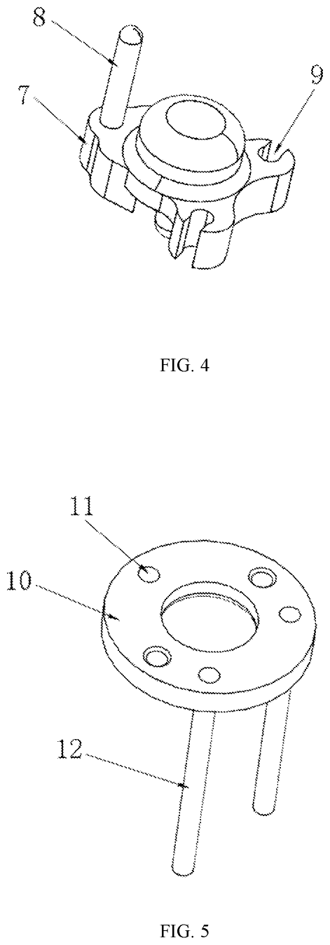

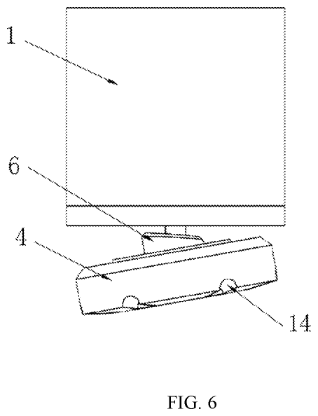

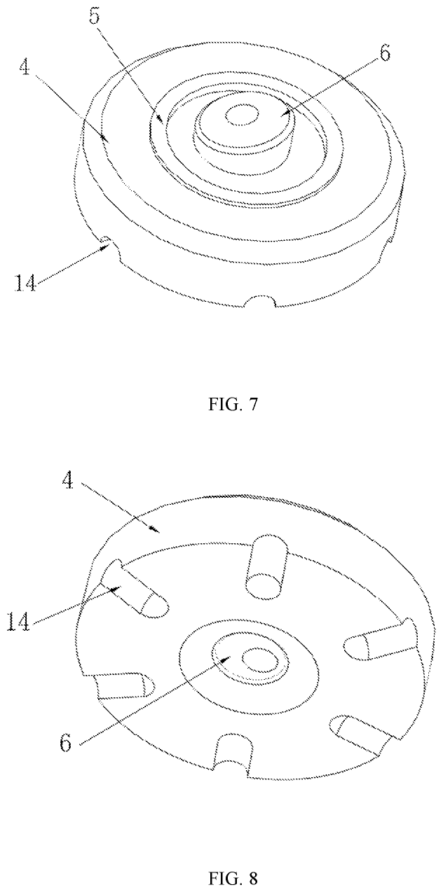

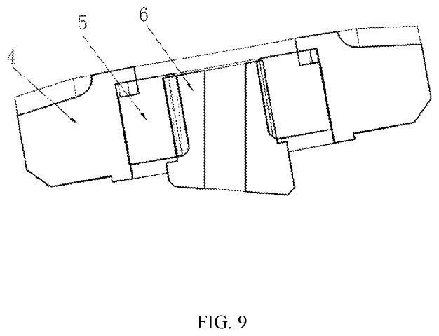

Referring to FIG. 1 to FIG. 9, the disclosure provides a tattoo marker, including a housing, a drive motor 1, a transmission mechanism and a tattoo needle, wherein the housing includes a handle part 2 and a drive part 3 which is detachably arranged on a top end of the handle part 2, the drive motor 1 is arranged inside the drive part 3, the transmission mechanism is arranged inside the handle part 2, the tattoo needle has a top end detachably connected to a head end of the handle part 2, the transmission mechanism is connected between the drive motor 1 and the tattoo needle, and the drive motor 1 drives the tattoo needle to extend longitudinally through the transmission mechanism. In existing tattoo markers, the transmission mechanisms generally are arranged inside the drive part 3; the transmission mechanism has a bottom end extending downwards to the handle part 2 for contact with the tattoo needle so as to drive the tattoo needle to extend, such arrangement results in a long space occupied by the transmission mechanism, consequently leading to a long tattoo marker, which is not convenient to operate, furthermore, the transmission mechanism will increase the noise generated during operation. However, according to the tattoo marker provided in the disclosure, the transmission mechanism is arranged inside the handle part 2, which makes the whole transmission mechanism moved downwards and consequently makes the drive motor in connection with the transmission mechanism moved downwards too, thereby shortening the distance between the drive motor 1 and the tattoo needle, shortening the length of the transmission mechanism, and shortening the overall length of the tattoo marker so that it is more convenient to hold the tattoo marker; furthermore, the noise generated during operation will be reduced alter the transmission mechanism is shortened.

In one preferred embodiment, the above transmission mechanism includes a faceplate 4, a sliding support and a sliding member, the drive motor 1 has an output shaft arranged downwards vertically, the faceplate 4 may be of a disc structure, the faceplate 4 is arranged obliquely, that is to say, an angle exists between an axial line of the faceplate 4 and an axial line of the output shaft, a mount hole is defined at the center of the faceplate 4, a bearing 5 is arranged in the mount hole, and the bearing 5 is in connection with the output shaft in a transmission way through a shaft sleeve 6, wherein central lines of the hearing 5 and the shaft sleeve 6 are collinear and they are collinear with the axial line of the faceplate 4, a shaft hole is defined on the shaft sleeve 6 for connection to the output shaft, an axial line of the shaft hole is along the vertical direction and is colinear with the axial line of the output shaft, the above sliding support is detachably arranged inside the handle part 2, the sliding member is arranged on the bottom of the sliding support and is sliding connection with the sliding support in vertical direction, the sliding member has a top in contact connection with the faceplate 4 in a locking way, and the sliding member has a bottom in contact connection with the tattoo needle. When in use, the drive motor 1 is started, the output shaft is rotated, the top of the sliding member is in contact connection with the faceplate 4 in a locking way, and the output shaft drives the shaft sleeve 6 to rotate; since an angle exists between the axial line of the shaft sleeve 6 and the axial line of the shaft hole at the center, when the shaft sleeve 6 is rotated within the bearing 5, any position of the faceplate 4 will reciprocate up and down, for example, when watching positions on two ends of any diameter of the faceplate 4, and taking a diameter perpendicular to this diameter as an axis, the two ends of the faceplate 4 swing up and down, like a seesaw; the sliding member is in contact connection with a position on the edge of the faceplate 4 in a locking way, and under the driving of the faceplate 4, the sliding member moves up and down inside the sliding support, thereby driving the tattoo needle to move up and down.

In one embodiment, the above sliding member includes a connection plate 7, a contact rod 8 and a first sliding hole 9, the connection plate 7 may be of a circle shape, a rectangle shape, a triangle shape or any other shapes, the contact rod may be arranged on one side of the top of the connection plate 7, a plurality of first sliding holes 9 may be defined, the contact rod 8 and the plurality of first sliding holes 9 may distributed along a circumferential direction of the connection plate 7, the contact rod 8 extends upwards, the first sliding hole 9 runs through the connection plate 7, the sliding support includes a support plate 10, the support plate 10 defines a second sliding hole 11 for the contact rod 8 to pass through at a position corresponding to the contact rod 8, a top end of the contact rod 8 passes through the second sliding hole 11 from a bottom end of the support plate 10 and is in sliding connection with the second sliding hole 11, the support plate 10 is provided with a sliding rod 12 at a position corresponding to the first sliding hole 9, the sliding rod 12 passes through the first sliding hole 9 from a top end of the connection plate 7, and the sliding rod 12 is in sliding connection with the first sliding hole 9; the above connect rod 8 and the first sliding hole 9, the sliding rod 12 and the second sliding hole 11 are all equivalent to guide devices, and multiple guide devices make the sliding action more stable and the work more reliable and also can reduce noises effectively.

Specifically, two first sliding holes 9 and two sliding rods 12 are provided, one contact rod 8 and one second sliding hole 11 are provided, the contact rod 8 passes through the second sliding hole 11 to extend upwards for a contact connection with the faceplate 4 in a locking way, so as to achieve a guide function; the first sliding hole 9 and the sliding rod 12 totally achieve a guide function; as a connection plate 7, a regular triangle may be formed by connecting points where the contact rod 8 and the centrals axial lines of the two first sliding holes 9 intersect with one same horizontal plane; or as a support plate 10, a regular triangle may be formed by connecting points where the two sliding rods 12 and the centrals axial line of the second sliding hole 11 intersect with one same horizontal plane; the regular triangle makes stress more balance and operation more stable.

Further, a bottom center of the sliding member is further provided with an ejector rod 13 extending downwards, and when the tattoo needle is in connection with the handle part 2, the top of the tattoo needle is in contact connection with a bottom end of the ejector rod 13.

It should be noted that the above faceplate 4 can push the sliding member to move downwards only when the faceplate moves down during the up down swing process, and the faceplate 4 cannot drive the sliding member to move upwards; the sliding member moves upwards under the drive of the tattoo needle, an elastic element is arranged between the top of the tattoo needle and the ejector rod 13; when the position of the faceplate 4 contacting the sliding member moves downwards, the sliding member is pushed to move downwards, thus the ejector rod 13 pushes the tattoo needle down, then the elastic part is compressed to accumulate an elastic force; when the position of the faceplate 4 contacting the sliding member moves upwards, the elastic part restores from deformation and releases the elastic force, thereby pushing the sliding member to move upwards, so that the sliding member keeps tight contact with the faceplate 4; therefore, a reciprocated up down slide of the sliding member is achieved, which thereby drives the tattoo needle to extend to and fro.

Specifically, the above elastic part may be a spring, preferably a rubber mat, which can effectively reduce noises.

Further, at least one locking groove 14 is defined on a bottom edge of the faceplate 4, and a plurality of the locking grooves 14 are evenly distributed along a circumferential direction of the faceplate 4 and extending along a radial direction of the faceplate 4; when the tattoo needle is not installed in the tattoo marker and when the tattoo marker is placed vertically, the sliding member is at a lowest position under the action of gravity, meanwhile the top end of the ejector rod 8 does not contact the faceplate 4; when the tattoo needle is installed, the tattoo needle pushes the sliding member up so that the contact rod 8 contacts the faceplate 4, meanwhile the contact rod 8 contacts any position of the faceplate 4, maybe a position defining a locking groove 14, or maybe a position not defining a locking groove 14; when the drive motor 1 is started, if the contact rod 8 is in the locking groove 14, the faceplate 4 does not rotate, but swings up and down simply; if the contact rod is at a position of the faceplate 4 not defining a locking groove 14, the faceplate 4 rotates when the drive motor 1 is started; when the faceplate 4 rotates until the contact rod 8 is clamped in the locking groove 14, the faceplate 4 stops rotating; while the faceplate 4 swings, the top end of the contact rod 8 will slide in a radial direction of the faceplate 4 within the locking groove 14.

Specifically, the top end of the contact rod 8 may be provided in a ball structure, and a cross section of the locking groove 14 may be provided in a circular arc structure, such that the top end of the contact rod 8 can slide into the locking groove 14 more smoothly.

The above drive motor 1 may be an electric machine or a pneumatic motor.

The above are the specific embodiments of the disclosure, merely; however, the protection scope of the disclosure is not limited to this any variations or substitutions easily thought by one skill familiar with the technical field within the technical scope of the disclosure are intended to be included within the scope of protection of the disclosure. Therefore, the scope of protection of the disclosure should be based on the protection scope of claims appended herein.

What is claimed is:

1. A tattoo marker, comprising a housing, a drive motor (1), a transmission mechanism and a tattoo needle, wherein the housing comprises a handle part (2) and a drive part (3) which is detachably arranged on a top end of the handle part (2), the drive motor (1) is arranged inside the drive part (3), the transmission mechanism is arranged inside the handle part (2), the tattoo needle is detachably arranged on a head end of the handle part (2), the transmission mechanism is connected between the drive motor (1) and the tattoo needle, and the drive motor (1) drives the tattoo needle to extend longitudinally through the transmission mechanism.

2. The tattoo marker according to claim 1, wherein the transmission mechanism comprises a faceplate (4), a sliding support and a sliding member, the drive motor (1) has an output shaft arranged downwards vertically, an angle is set between an axial line of the faceplate (4) and an axial line of the output shaft, and a bearing (5) is arranged at a center of the faceplate (4), the bearing (5) is in connection with the output shaft through a shaft sleeve (6), both axial lines of the shaft sleeve (6) and the bearing (5) are collinear with the axial line of the faceplate (4), an axial line of a shaft hole defined on the shaft sleeve (6) is colinear with the axial line of the output shaft, the sliding support is detachably arranged inside the handle part (2), the sliding member is arranged on the bottom of the sliding support and is sliding connection with the sliding support in vertical direction, the sliding member has a top in contact connection with the faceplate (4) in a locking way, and the sliding member has a bottom in contact connection with the tattoo needle.

3. The tattoo marker according to claim 2, wherein the sliding member comprises a connection plate (7), a contact rod (8) arranged on an edge of the connection plate (7) and a plurality of first sliding holes (9), the contact rod (8) and the plurality of first sliding holes (9) are distributed along a circumferential direction of the connection plate (7), the contact rod (8) is arranged on a top of the sliding member and extends upwards, the first sliding hole (9) runs through the connection plate (7), the sliding support comprises a support plate (10), the support plate (10) defines a second sliding hole (11) for the contact rod (8) to pass through at a position corresponding to the contact rod (8), the support plate (10) is provided with a sliding rod (12) at a position corresponding to the first sliding hole (9), and the sliding rod (12) is in sliding connection with the sliding hole (9).

4. The tattoo marker according to claim 3, wherein a bottom center of the sliding member is further provided with an ejector rod (13) extending downwards for contact with the tattoo needle.

5. The tattoo marker according to claim 3, wherein at least one locking groove (14) is defined on a bottom edge of the faceplate (4), a plurality of the locking grooves (14) are distributed along a circumferential direction of the faceplate (4) and extending along a radial direction of the faceplate (4), and a top end of the contact rod (8) is in contact connection with the locking groove (14).

6. The tattoo marker according to claim 3, wherein two first sliding holes (9) and two sliding rods (12) are provided, the contact rod (8) and the first sliding holes (9) form a regular triangle around a center point of one same horizontal plane, or the second sliding hole (11) and the sliding rods (12) form a regular triangle around a center point of one same horizontal plane.

7. The tattoo marker according to claim 4, wherein an elastic piece is arranged where a top of the tattoo needle contacts the ejector rod (13).

8. The tattoo marker according to claim 7, wherein the elastic piece is a rubber mat.

9. The tattoo marker according to claim 5, wherein the top end of the contact rod (8) is in a ball shape, and a cross section of the locking groove (14) is in a circular arc shape.

10. The tattoo marker according to claim 1, wherein the drive motor (1) is an electric machine or a pneumatic motor.
